(19) United States
(12) Patent Application Publication
(10) Pub. No.: US 2026/0120880 A1
(43) Pub. Date: Apr. 30, 2026

(54) HEALTH MANAGEMENT SYSTEM AND METHOD THROUGH REAL-TIME CARDIOVASCULAR CONDITION MONITORING AND PREDICTION

(71) Applicant: GREBT Co., Ltd., Sejong-si (KR)

(72) Inventor: Hang Joo CHOI, Cheonan-si (KR)

(73) Assignee: GREBT Co., Ltd., Sejong-si (KR)

(21) Appl. No.: 18/952,306

(22) Filed: Nov. 19, 2024

(30) Foreign Application Priority Data
Oct. 25, 2024 (KR) 10-2024-0147483

(51) Int. Cl. G16H 50/30; A61B 5/00; A61B 5/0205; A61B 5/024; A61B 5/11; A61B 5/363; G16H 10/60

(52) U.S. Cl. CPC G16H 50/30; A61B 5/02055; A61B 5/1118; A61B 5/363; A61B 5/4809; A61B 5/6822; A61B 5/7275; A61B 5/742; G16H 10/60; A61B 5/024

(57) ABSTRACT

Disclosed is a cardiovascular health management system including a communication module that receives biometric information of a user including information about a heartbeat, and a processor that generates cardiovascular monitoring and prediction information based on the received biometric information. The processor analyzes the biometric information and records a time at which an abnormal pulse is detected, heartbeat information at a corresponding time, and state information of the user when the abnormal pulse is detected, provides a user terminal with a number of times that the abnormal pulse occurs during a predetermined time, and provides a phased notification to the user terminal depending on the number of times that the abnormal pulse occurs.

### CROSS-REFERENCE TO RELATED APPLICATIONS

A claim for priority under 35 U.S.C. § 119 is made to Korean Patent Application No. 10-2024-0147483 filed on Oct. 25, 2024 in the Korean Intellectual Property Office, the entire contents of which are hereby incorporated by reference.

### BACKGROUND

Embodiments of the present disclosure described herein relate to a system capable of monitoring a cardiovascular condition in real time and evaluating the cardiovascular condition.

Nowadays, a digital healthcare market is significantly growing every year.

However, while this market is growing, the accuracy of analyzing a user's health status in real time is still lacking.

In particular, a technology for analyzing the user's biometric information to monitor a cardiovascular condition and evaluating the cardiovascular condition is inaccurate and has long delays in providing determination results.

Accordingly, there is a need for a technology for analyzing the user's biometric information in real time to monitor the cardiovascular condition and accurately evaluating the cardiovascular condition.

### SUMMARY

Embodiments of the present disclosure provide a system for monitoring a cardiovascular condition.

Moreover, embodiments of the present disclosure provide a system for evaluating a user's cardiovascular condition.

Furthermore, embodiments of the present disclosure recommend a rest or provide a notification depending on the frequency of abnormal pulse detected from biometric information collected from the user.

Besides, embodiments of the present disclosure calculate and provide a cardiovascular health index of the user based on biometric information collected from the user.

Also, embodiments of the present disclosure analyze the user's cardiovascular condition based on the user's cardiovascular scale information and basic information, and generate and provide the user's customized health information based on the user's cardiovascular condition.

Problems to be solved by the present disclosure are not limited to the problems mentioned above, and other problems not mentioned will be clearly understood by those skilled in the art from the following description.

According to an embodiment, a cardiovascular health management system includes a communication module that receives biometric information of a user including information about a heartbeat, and a processor that generates cardiovascular monitoring and prediction information based on the received biometric information. The processor analyzes the biometric information and records a time at which an abnormal pulse is detected, heartbeat information at a corresponding time, and state information of the user when the abnormal pulse is detected, provides a user terminal with a number of times that the abnormal pulse occurs during a predetermined time, and provides a phased notification to the user terminal depending on the number of times that the abnormal pulse occurs.

According to an embodiment, a control method performed by a processor of a cardiovascular health management system includes receiving biometric information of a user including information about a heartbeat, analyzing the biometric information and recording a time at which an abnormal pulse is detected, heartbeat information at a corresponding time, and state information of the user when the abnormal pulse is detected, providing a user terminal with a number of times that the abnormal pulse occurs during a predetermined time, and providing a phased notification to the user terminal depending on the number of times that the abnormal pulse occurs.

Besides, a computer program stored in a computer-readable recording medium for executing a method to implement the present disclosure may be further provided.

In addition, a computer-readable recording medium for recording a computer program for performing the method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a schematic diagram of a health management system through cardiovascular condition monitoring and prediction, according to an embodiment of the present disclosure;

FIG. 2 is a block diagram of an electronic device, according to an embodiment of the present disclosure;

FIG. 3 is a block diagram of a server that monitors cardiovascular conditions and evaluates states, according to an embodiment of the present disclosure;

FIG. 4 is a perspective view of the electronic device of FIG. 1;

FIG. 5 is an example diagram of an electronic device including a sensor module;

FIG. 6 is a diagram illustrating that a user wears an electronic device;

FIG. 7 is a flowchart of a cardiovascular condition monitoring method as a cardiovascular health managing method, according to an embodiment of the present disclosure;

FIGS. 8A and 8B are examples of an abnormal pulse;

FIG. 9 is a flowchart of a cardiovascular condition evaluating method as a cardiovascular health managing method, according to an embodiment of the present disclosure; and

FIG. 10 is a diagram illustrating state monitoring information provided to a user terminal.

### DETAILED DESCRIPTION

The same reference numerals denote the same elements throughout the present disclosure. The present disclosure does not describe all elements of embodiments. Well-known content or redundant content in which embodiments are the same as one another will be omitted in a technical field to which the present disclosure belongs. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

In this specification, ‘an electronic device or a server according to an embodiment of the present disclosure’ includes all various devices capable of providing results to a user by performing arithmetic processing. For example, the electronic device according to an embodiment of the present disclosure may include all of a computer, a server, and a portable terminal, or may be in any one form.

Here, for example, the computer may include a notebook computer, a desktop computer, a laptop computer, a tablet PC, a slate PC, and the like, which are equipped with a web browser.

The server may be a server that processes information by communicating with an external device and may include an application server, a computing server, a database server, a file server, a game server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet terminal (Wibro) terminal, and a wearable device such as a timepiece, a ring, a bracelet, an anklet, a necklace, glasses, a contact lens, or a head-mounted device (HMD).

Hereinafter, an embodiment of the present disclosure will be described in detail with reference to the accompanying drawings.

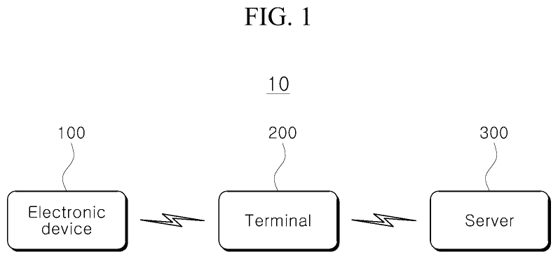

FIG. 1 is a schematic diagram of a health management system through cardiovascular condition monitoring and prediction, according to an embodiment of the present disclosure.

Referring to FIG. 1, a health management system 10 for monitoring and predicting cardiovascular conditions according to an embodiment of the present disclosure includes an electronic device 100, a terminal 200, and a server 300.

However, in some embodiments, the system 10 may include fewer or more components than the components illustrated in FIG. 1.

The electronic device 100 refers to a device for monitoring cardiovascular conditions.

To this end, the electronic device 100 includes a processor 110, a communication module 120, a memory 130, and a sensor module 140. The sensor module 140 senses at least one of information about interiors of the present device, surrounding environmental information surrounding the present device, and user information and generates a sensing signal corresponding to the sensed result. On the basis of the sensing signal, the processor 110 may control the driving or operation of the present device or may perform the data processing, function, or operation associated with an application program installed in the present device.

The sensor module 140 may include at least one of a proximity sensor, an illumination sensor, a touch sensor, an acceleration sensor, a magnetic sensor, a G-sensor, a gyroscope sensor, a motion sensor, an RGB sensor, an infrared sensor (IR sensor), a finger scan sensor, an ultrasonic sensor, an optical sensor (e.g., a camera), a microphone, an environmental sensor (e.g., including at least one of a barometer, a hygrometer, a thermometer, a radiation detection sensor, a heat detection sensor, and a gas detection sensor), and a chemical sensor (e.g., a healthcare sensor, a biometric sensor, or the like). In the meantime, the present device may combine and utilize pieces of information sensed by at least two or more of these sensors.

In an embodiment of the present disclosure, the sensor module 140 includes an electrode, and may also include other sensors in addition to the electrode according to an embodiment.

The sensor module 140 may noninvasively measure at least one of, but not limited to, an electrocardiogram (ECG), blood pressure, blood sugar, pulse, oxygen saturation, blood flow, respiration rate, body temperature, step count, and electrode temperature.

The electronic device 100 may be applied to any computing device that includes a control means (e.g., a processor or a control unit) and a communication means. The terminal 200 may be a means for communicating and displaying various pieces of information generated by the electronic device 100. Any computing device including a control means, a communication means, and an output means, such as a smartphone, tablet PC, or laptop PC may be applied to the terminal 200.

Moreover, the terminal 200 may also transmit data received from the electronic device 100 to the server 300.

The server 300 is a device that performs health management through real-time cardiovascular condition monitoring and prediction. The server 300 receives information from the electronic device 100, the terminal 200, and an external device, analyzes the information to managing health by monitoring and predicting cardiovascular conditions in real time, and provides the analysis information to the terminal 200.

As long as the server 300 is a computing device including a control means (a processor) and a communication means, there is no limitation on the server 300.

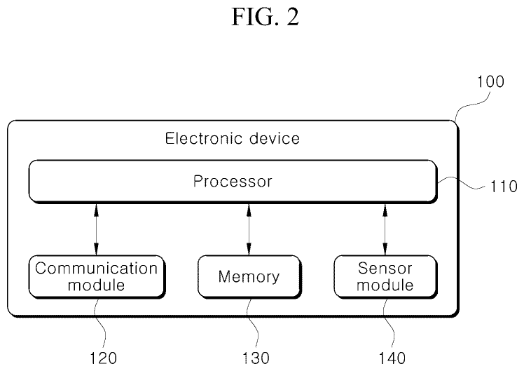

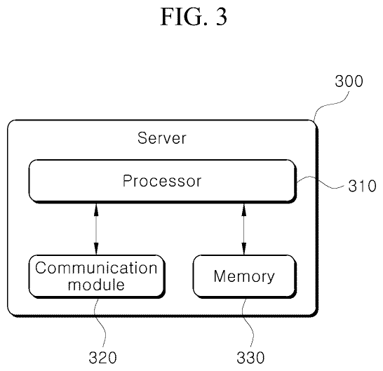

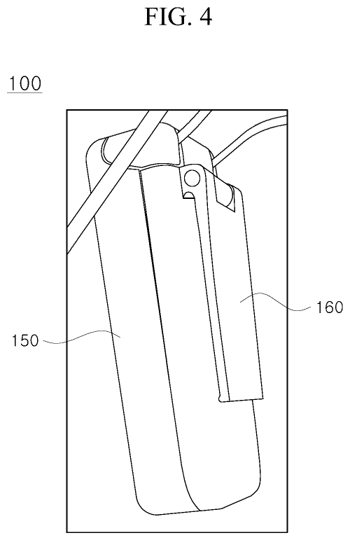

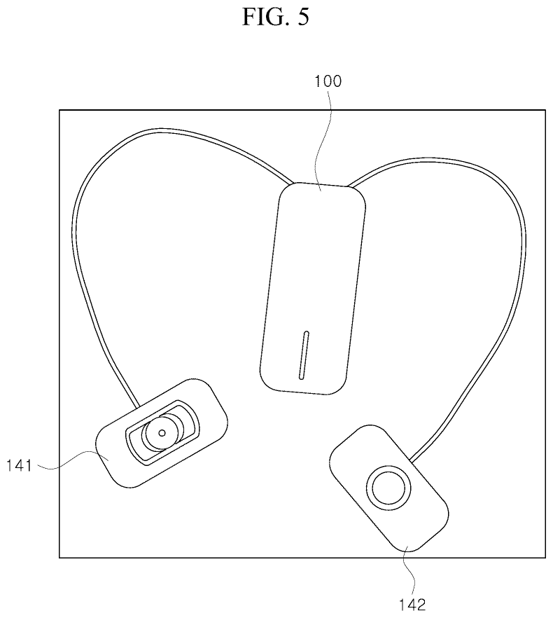

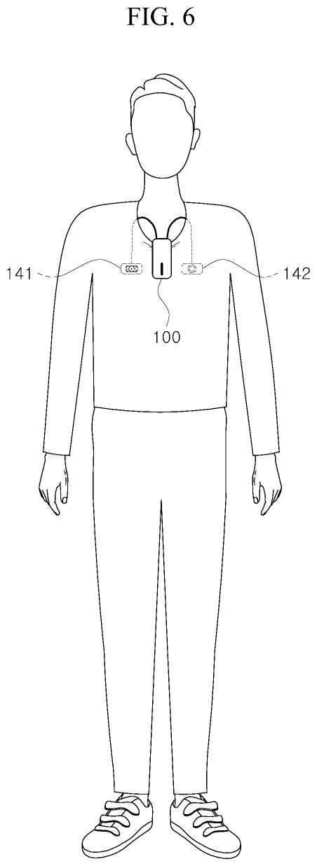

FIG. 2 is a block diagram of an electronic device, according to an embodiment of the present disclosure. FIG. 3 is a block diagram of a server that monitors cardiovascular conditions and evaluates states, according to an embodiment of the present disclosure. FIG. 4 is a perspective view of the electronic device of FIG. 1. FIG. 5 is an example diagram of an electronic device including a sensor module. FIG. 6 is a diagram illustrating that a user wears an electronic device.

Referring to FIG. 3, the server 300 includes a processor 310, a communication module 320, and a memory 330.

However, in some embodiments, the server 300 may include fewer or more components than the components illustrated in FIG. 2.

The processor 310 may be implemented with the memory 330 that store data regarding an algorithm for controlling operations of components within the present device, or a program for realizing the algorithm, and the at least one processor 310 that perform the above-described operation by using the data stored in the memory 330. In this case, each of the memory 330 and the processor 310 may be implemented as separate chips. Alternatively, the memory 330 and the processor 310 may be implemented as a single chip.

Furthermore, to implement various embodiments of the present disclosure described below in the drawing, the processor 310 may control one of the components described above or the combination of the components.

In addition to an operation associated with the application program, the processor 310 may generally control overall operations of the present device. The processor 310 may provide or process appropriate information or functions to a user, by processing a signal, data, information, or the like, which is input or output through the above-described components, or driving the application program stored in the memory.

Besides, the processor 310 may control at least part of the components of the present device to operate the application program stored in the memory 330. Furthermore, the processor 310 may combine and operate at least two or more of the components included in the present device to operate the application program.

The processor 310 may be implemented as one or more processors. Hereinafter, the processor 310 may be considered as a plurality of processors even though it is expressed as a single processor. The processor 310 may control configurations of a server. The processor 310 may refer to a data processing device, which has a physically structured circuit to perform functions expressed by codes or instructions included in a program and which is built into hardware. As such, the processor 310 may be an example of a data processing device built into hardware, and may include the microprocessor, a central processing device (CPU), a processor core, the multiprocessor, an application-specific integrated circuit (ASIC), a field programmable gate array (FPGA), or the like, but the scope of the present disclosure is not limited thereto. The processor 310 may be separately equipped with the running processor for performing artificial intelligence computations, or may be equipped with the running processor in itself.

In various embodiments, the processor 310 may include one or more of a central processing unit (CPU), an application processor (AP), or a communication processor (CP). At least part of the processor may be hardware, and may access a memory and may perform functions related to instructions stored in the memory.

The communication module 320 may include one or more modules connecting the server 300 to one or more networks.

The communication module 320 may include one or more components capable of communicating with an external device, and may include, for example, at least one of a broadcast reception module, a wired communication module, a wireless communication module, a short-range communication module, and a location information module.

Here, in addition to various wired communication modules such as a Local Area Network (LAN) module, a Wide Area Network (WAN) module, or a Value Added Network (VAN) module, the wired communication module may include a variety of cable communication modules such as Universal Serial Bus (USB), High Definition Multimedia Interface (HDMI), Digital Visual Interface (DVI), recommended standard232 (RS-232), power line communication, or plain old telephone service (POTS).

Here, the wireless communication module may include a wireless communication module for supporting various wireless communication methods such as Global System for Mobile (GSM) communication, Code Division Multiple Access (CDMA), Wideband Code Division Multiple Access (WCDMA), Universal Mobile Telecommunication System (UMTS), Time Division Multiple Access (TDMA), Long Term Evolution (LTE), 4G, 5G, and 6G in addition to a Wi-Fi module and Wireless broadband module.

The wireless communication module may include a wireless communication interface including an antenna and a transmitter that transmit communication signals. Moreover, the wireless communication module may further include a signal conversion module that modulates a digital control signal, which is output from the processor through a wireless communication interface, into an analog wireless signal under the control of the processor.

The short-range communication module may be used for short range communication, and may support short-range communication by using at least one of Bluetooth™, radio frequency identification (RFID), infrared data association (IrDA), ultra wideband (UWB), ZigBee, near field communication (NFC), Wireless-Fidelity (Wi-Fi), Wi-Fi Direct, and wireless universal serial bus (Wireless USB) technologies.

The communication module 320 may also use the name of the communication interface.

The communication module 320 may establish communication between the electronic device and an external device. For example, the communication interface may communicate with the external device through wireless communication (e.g., Wireless Fidelity (Wi-Fi), Bluetooth, Near Field Communication (NFC), magnetic stripe transmission (MST), or the like) or wired communication.

The memory 330 may store data for supporting various functions of the present device. The memory may store a plurality of application programs (or applications) running in the present device, data for an operation of the present device, and instructions. At least part of the application programs may be present for basic functions of the present device. In the meantime, the application program may be stored in the memory, may be installed in the device, and may be driven by the processor so as to perform an operation.

The memory 330 may store data for supporting various functions of the present device, and a program for operations of the processor 110, may store pieces of input/output data (e.g., music files, still images, videos, and the like), and may store a plurality of application programs (or applications) running on the present device, pieces of data for operations of the present device, and instructions. At least part of the application programs may be downloaded from an external server through wireless communication.

The memory 330 may include the type of a storage medium of at least one of a flash memory type, hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory may be separate from the present device, but may be a database connected by wire or wirelessly.

The memory 330 may be electrically connected to the processor and may store at least one code executed by the processor. The memory may refer to various types of storage devices. The memory may store information needed to perform computations by using artificial intelligence, machine learning, and artificial neural networks.

The memory 330 may store various learning models. The learning models stored in the memory may infer result values for new input data, not learning data. The inferred values may be used as the determination basis for performing specific operations. The learning models stored in the memory may be learned based on label information. To increase the accuracy of learning, various backpropagation algorithms may be applied to the learning models such that a loss function has a target value.

Besides, the memory 330 may include a plurality of processes for controlling the server. Furthermore, the server 300 may further include a battery for driving the server 300.

In the meantime, the above description of the processor 310, the communication module 320, and the memory 330 may also be applied to the processor 110, the communication module 120 and the memory 130.

The configuration of the electronic device 100 illustrated in FIG. 2 is a configuration related to the operation process, and FIGS. 4 and 5 illustrate the external shape of the electronic device.

Referring to FIGS. 4 and 5, the electronic device 100 includes a body 150, a clip 160, and electrodes 141 and 142.

The body 150 is a configuration in which other components in addition to the electrodes 141 and 142 are built. The body 150 may include a power button on a side.

The electrodes 141 and 142 are configurations of a sensor module.

The electrodes 141 and 142 are illustrated as two in the drawing, but are not limited thereto. For example, the sensor module may include at least one electrode.

Moreover, the electrodes 141 and 142 are connected to the body 150 by wire or wirelessly, and thus the processor 310 may receive values measured through the electrodes 141 and 142.

The clip 160 may be formed in a hanging shape on the rear of the body 150, and the clip 160 may be connected to the body 150 through an elastic member.

The electronic device 100 may further include a wearable-type necklace, and may be prevented from falling off the user's body by using at least one of the clip 160 and the necklace.

Referring to FIG. 6, the body 150 may be fixed to a neckline of the user's upper garment through an elastic member of the clip 160.

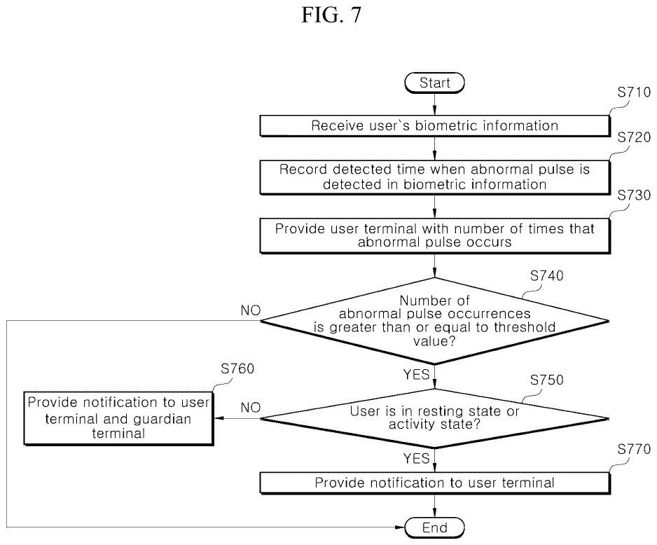

FIG. 7 is a flowchart of a cardiovascular condition monitoring method as a cardiovascular health managing method, according to an embodiment of the present disclosure.

A process for monitoring a cardiovascular condition according to an embodiment of the present disclosure will be described with reference to FIG. 7.

The processor 310 receives a user's biometric information. (S710) In more detail, the server 300 may receive biometric information of the user measured through the sensor module 140 of the electronic device 100 and may also receive biometric information from an external device other than the electronic device 100.

The processor 310 analyzes the user's biometric information in real time or at a predetermined time.

In an embodiment of the present disclosure, the biometric information includes heartbeat information. Here, the heartbeat information may be understood as a concept including pulse information. In some embodiments, the biometric information may further include at least one of blood pressure information, blood sugar information, body fat percentage information, cholesterol level information, age information, weight information, respiratory rate information, exercise information, and body temperature information (electrode temperature information).

In an embodiment, the biometric information may be received from a user terminal or may be a value measured in real time through the sensor module 140. For example, the biometric information such as the user's age information and weight information may be entered into the user terminal and may be received, and the biometric information such as heartbeat information and pulse information may be measured and received through the sensor module 140.

When an abnormal pulse is detected in the biometric information by analyzing the biometric information, the processor 310 records the time at which the abnormal pulse is detected. (S720) Here, the abnormal pulse may be understood as a concept that includes both an abnormal heartbeat and an abnormal pulse, and may include various pieces of information measured in relation to a heartbeat.

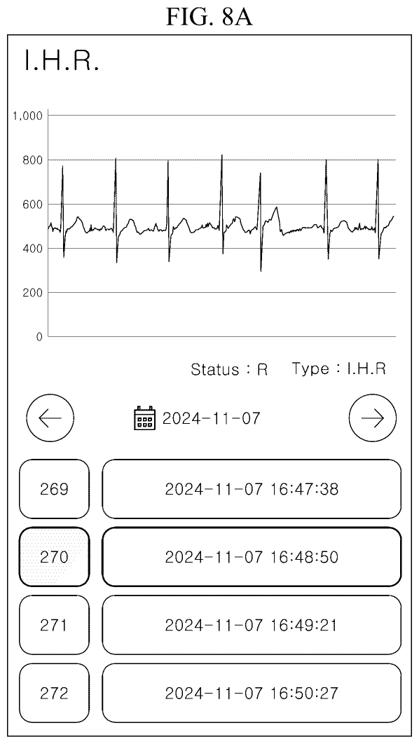

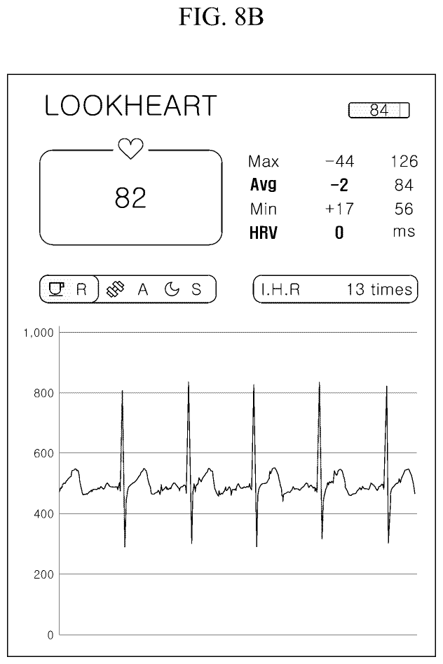

For example, there may be an abnormal pulse, such as an arrhythmia. Here, the abnormal pulse is due to an irregular heartbeat rate, which means that the rhythm of the heartbeat is irregular. In this case, the pulse may seem to be skipped, or may appear to be irregular. Referring to FIG. 8A, abnormal pulses may be identified. Referring to FIG. 8B, continuous abnormal pulses may be identified.

In an embodiment, the memory 330 may store criteria for the range, pattern, and regularity of normal pulses, and data regarding the normal pulses may be data optimized for a user.

In the meantime, the processor 310 may use a trained machine learning model to detect abnormal pulses. In some embodiments, the processor 310 may create and utilize a personalized prediction model by continuously collecting and analyzing the corresponding information.

In some embodiments, the processor 310 may receive feedback that the user feels an abnormal pulse, from the user through a user terminal. That is, the user may enter information about a time point, at which he/she feels abnormal pulses, and symptoms of abnormal pulses into the user terminal. At a point in time when the user feels the abnormal pulse, the processor 310 determines whether the processor 310 detects that an abnormal pulse occurs. When the processor 310 does not detect the occurrence of an abnormal pulse at the corresponding time point, the processor 310 may store heartbeat information of a time point, at which the user feels the abnormal pulse, may analyze a pattern, and may update a model for detecting the abnormal pulse.

In the meantime, the processor 310 may analyze biometric information, and may record the heartbeat information and the user's state information at the corresponding time in addition to a time point, at which the abnormal pulse is detected, when an abnormal pulse is detected. Accordingly, when the user selects the time point, at which an abnormal pulse is detected, through the user terminal, the user may identify the heartbeat information and the state information at the corresponding time together.

Here, the processor 310 determines the user's state by analyzing the biometric information. The user's state may be determined as one of a resting state, an activity state, and a sleep state, which will be described in detail later.

The processor 310 provides the user's terminal with the number of times that an abnormal pulse occurs. (S730)

The processor 310 allows the user to identify the number of abnormal pulse occurrences, which is accumulated during a predetermined time. However, when the predetermined time has elapsed, the processor 310 may reset the accumulated number of abnormal pulse occurrences. The reset period may be one hour or one day, but is not limited thereto.

When the number of abnormal pulse occurrences is within an allowable range, the processor 310 provides only the accumulated number and does not provide a separate notification. However, when the number of times that an abnormal pulse occurs exceeds the allowable range, the processor 310 may provide guide information based on the number of times. That is, the processor 310 provides a phased notification to the user terminal depending on the number of times that an abnormal pulse occurs, which will be described later. The processor 310 determines whether the number of abnormal pulse occurrences is greater than or equal to a threshold value. (S740) The memory 330 may store a plurality of threshold values for the number of abnormal pulse occurrences. For example, the accumulated number of abnormal pulse occurrences may be 10, 20, 30 and 50 times or 50 and 100 times, but is not limited thereto.

In an embodiment, the processor 310 provides the phased notification to the user terminal for a plurality of predetermined counts for abnormal pulses. Because the content included in the notification is changed as the number of abnormal pulse occurrences increases, it is necessary to identify a threshold value, which the number of abnormal pulse occurrences exceeds, from among the plurality of threshold values.

When the number of abnormal pulse occurrences is greater than or equal to the threshold value, the processor 310 identifies the user's state. (S750)

The processor 310 determines the user's state by analyzing the biometric information. The user's state is determined to be one of a resting state, an activity state, and a sleep state. In an embodiment, the processor 310 may determine the state of a user based on a pattern of the biometric information.

In some embodiments, the processor 310 may receive a resting heart rate of the user, which is measured while the user is at rest, and an activity heart rate of the user measured while the user is moving. To this end, the processor 310 may request the user to rest for a predetermined time, and then to walk continuously for a predetermined time.

Moreover, the processor 310 may set an activity reference heart rate based on the measured resting heart rate and the measured activity heart rate, and the activity reference heart rate may be set for each user. For example, the activity reference heart rate may be set between the average of the resting heart rate and the average of the activity heart rate, but is not limited thereto.

Accordingly, the processor 310 may analyze the user's biometric information and may determine the user's state as an activity state when the heart rate is greater than or equal to the activity reference heart rate.

Besides, the biometric information may include step count information. When it is determined that the user is not moving because a step count does not increase, the processor 310 may determine the user's state as a resting state. In some embodiments, the processor 310 may analyze the user's biometric information and may determine the user's state as a resting state when the heart rate is less than the activity reference heart rate.

In the meantime, the user may set a sleep time through the user terminal. When a current time corresponds to the sleep time entered by the user, the processor 310 may determine the user's state as a sleep state.

When the user is in a resting state or an activity state, the processor 310 provides a notification to the user terminal (S770). In S750, when the user is in a sleep state, the processor 310 provides the user terminal and a guardian terminal with a notification that an abnormal pulse occurs. (S760)

In detail, when the number of abnormal pulses occurring during a predetermined time is greater than or equal to the predetermined count and the user's state is a resting state or an activity state, the processor 310 provides a notification through the user terminal. However, because it is difficult for the user to identify the notification when the user is in a sleep state, the processor 310 provides a notification to the guardian terminal as well as the user terminal when the number of abnormal pulses occurring during the predetermined time is greater than or equal to the predetermined count and the user is in a sleep state.

As described above, the plurality of threshold values are stored with respect to the number of abnormal pulse occurrences. The processor 310 provides the user terminal with the phased notification with respect to abnormal pulses at a plurality of predetermined respective counts. However, as the number of abnormal pulses increases, the content included in the notification may change. As the number of abnormal pulses increases, the content of the notification may include immediate action instructions.

For example, when the accumulated number of abnormal pulses is 10 times, a light notification that you are careful of stress may be provided. However, when the accumulated number of abnormal pulses is 50 times, a notification that you should seek medical attention, or a notification that you should rest may be provided.

In the meantime, in some embodiments, when providing a rest recommendation notification to the user terminal, the processor 310 may include a message indicating that the user is asked to press an OK button when the user identifies the corresponding notification. When the processor 310 does not receive a signal, which indicates that the user has pressed the OK button within a specified time, from the user terminal, the processor 310 may determine that the user is in danger, and then may provide the guardian terminal with a notification that abnormal pulses occur.

In some embodiments, when the number of abnormal pulses occurring during a predetermined time is greater than or equal to the highest count among predetermined counts, the processor 310 may provide the user terminal with a notification for requesting an acknowledgement that the corresponding notification is acknowledged, together with a notification for recommending a rest. When the processor 310 does not receive an acknowledgement signal for the corresponding notification from the user terminal, the processor 310 may also provide a notification to the user's guardian terminal.

In the meantime, the processor 310 may analyze the biometric information and may provide a notification to the user terminal when the heart rate is greater than or equal to a predetermined tachycardia reference heart rate (e.g., greater than or equal to 100 beats per minute) and the heart rate is less than or equal to a predetermined bradycardia reference heart rate (e.g., less than or equal to 60 beats per minute).

However, tachycardia or bradycardia may not occur due to the user's health problems, but may occur as a normal physiological response during the user's activity. For example, the tachycardia may occur during exercise or caffeine intake, and the bradycardia may occur during sleep.

Accordingly, the processor 310 may determine a state of the user and may not alert the user or the guardian of danger when tachycardia or the bradycardia appears as a normal physiological response.

To this end, in some embodiments, when the heart rate is greater than or equal to the predetermined tachycardia reference heart rate but the user is in an activity state, and the heart rate is less than or equal to the predetermined bradycardia reference heart rate but the user is in a sleep state, the processor 310 may not provide a notification to the user terminal. That is, when the processor 310 determines that the user is in tachycardia but is in an activity state, or when the user is in bradycardia but is in a sleep state, the processor 310 may determine that an abnormal pulse has occurred as a normal physiological response, and may not provide a notification to the user terminal or the guardian terminal.

Besides, when the heart rate is greater than or equal to the predetermined tachycardia reference heart rate or the heart rate is less than or equal to the predetermined bradycardia reference heart rate, the processor 310 may send an inquiry notification to the user terminal to inquire about what state the user is in. The corresponding inquiry notification may be a notification for identifying a user state, and may be an inquiry notification about whether the corresponding tachycardia or bradycardia is in a state that may only occur as a normal physiological response.

For example, when the heart rate is greater than or equal to the predetermined tachycardia reference heart rate, the processor 310 may provide the user terminal with a notification to inquire about whether the user is exercising or consuming caffeine. When the heart rate is less than or equal to the predetermined bradycardia reference heart rate, the processor 310 may provide the user terminal with a notification to inquire about whether the user is sleeping.

When the processor 310 receives a positive response (a response indicating that the corresponding state is correct) from the user terminal, the processor 310 may not provide a risk notification to the user terminal or the guardian terminal. However, when the processor 310 receives a negative response (a response indicating that the corresponding state is not correct) from the user terminal, the processor 310 provides a risk notification to the user terminal or the guardian terminal.

In the meantime, the electronic device 100 may receive electrode temperature information through the communication module 320. The processor 310 may use the electrode temperature information as an auxiliary indicator.

When the number of abnormal pulses occurring during a predetermined time is greater than or equal to the predetermined count, and the electrode temperature information is out of a predetermined temperature range, the processor 310 may provide a notification to at least one of the user terminal and the guardian terminal.

In an embodiment, the processor 310 may determine the predetermined temperature range based on a plurality of body temperatures measured during the user's normal state.

In detail, when the electrode temperature is out of the predetermined temperature range even though the number of times that an abnormal pulse occurring during the predetermined time is greater than or equal to a count less than the highest count among the predetermined counts, the processor 310 may provide the user terminal with a notification for requesting an acknowledgement that the corresponding notification is acknowledged, together with a notification for recommending rest. When the processor 310 does not receive an acknowledgement signal for the corresponding notification from the user terminal, the processor 310 may also provide a notification to the user's guardian terminal.

That is, even though the number of abnormal pulse occurrences does not reach 50 times when a threshold value for the accumulated number of abnormal pulse occurrences is set to 10, 20, 30, and 50 times, in the case where an abnormality is identified as the number of abnormal pulse occurrences reaches 10, 20, or 30 times, and the electrode temperature is out of the predetermined temperature range, there is a high possibility that a problem has occurred in the cardiovascular system, the processor 310 may most powerfully cope with the cardiovascular problem.

The biometric information may further include step count information.

In an embodiment, the electronic device may include a gyroscope sensor or an acceleration sensor, and may measure the user's step count by using the gyroscope sensor or the acceleration sensor.

In an embodiment, the processor 310 may receive the measured step count through the user's terminal at a predetermined time.

In an embodiment, the processor 310 may be connected to a user's wearable device (e.g., a smart watch) through the communication module 320 and may receive the user's step count from the wearable device.

The processor 310 may calculate activity information including the user's activity calories and movement distance based on the user's step count, and may provide the activity information to the user terminal.

In an embodiment, when the number of abnormal pulses occurring during a predetermined time is greater than or equal to the predetermined number, the processor 310 may provide a notification to the user terminal through the communication module 320. In an embodiment, when no biometric information is received during a predetermined time, the processor 310 may provide the user terminal with a notification that the sensor module 140 is removed.

In detail, because the electrode is attached to the user's chest area, there are cases where the electrode may fall off. When no biometric information is received from the electrode for a predetermined time, the processor 310 may provide the user terminal a notification that the electrode is removed from the body.

In an embodiment, the processor 310 may calculate a user's cardiovascular health index based on \the amplitude of a pulse identified through the biometric information, the pattern of the pulse, and the number of abnormal pulses accumulated during the predetermined time.

Furthermore, the processor 310 may provide the generated cardiovascular health index to a user terminal.

In this case, the processor 310 may calculate the user's cardiovascular health index in further consideration of at least one of blood pressure information, blood sugar information, body fat percentage information, cholesterol level information, age information, weight information, respiratory rate information, exercise information, and body temperature information.

In an embodiment, the processor 310 may determine whether the user's cardiovascular health state is normal, based on the size of the waveform of the biometric information over time and the regularity of a model.

In an embodiment, the processor 310 may determine whether the user's cardiovascular health state is normal, based on first similarity between a first waveform of the biometric information, which is obtained at a current time point, and a second waveform of the biometric information immediately before the current time point. When the user's cardiovascular health state is determined to be abnormal based on the first similarity-based determination, the processor 310 may determine whether the user's cardiovascular health state is normal, based on the similarity between the waveform of the biometric information obtained at the current time point and ‘N’ previous waveforms.

At this time, the processor 310 may determine ‘N’ based on the first similarity. As the similarity decreases, ‘N’ may increase. In other words, the processor 310 may determine whether the user's cardiovascular health state is normal, by using the more previous waveforms as a difference between the first waveform and the second waveform is great.

In some embodiments, the processor 310 may calculate a cardiovascular health index based on the number of times abnormal pulses occurred during a predetermined time and additional information, the user's biometric information may further include at least one of blood pressure information, blood sugar information, body fat percentage information, cholesterol level information, age information, weight information, respiratory rate information, and exercise information as additional information, and may be derived through Equation 1 below, but is not limited thereto.

Cardiovascular health index=100−Number of abnormal pulses/10−Weight*Score according to additional information [equation 1]

Here, the weight may be a value for adjusting the importance according to additional information, and the score according to additional information may be a score assigned to a factor, which increases the risk of cardiovascular disease, for respective additional information. For example, with regard to the age information, 0 points may be assigned to those under 20 years old; 1 point may be assigned to those between 20 and 39 years old; 2 points may be assigned to those between 40 and 59 years old; and 3 points may be assigned to those over 60 years old.

In some embodiments, the processor 310 may adjust the highest count among predetermined counts set for abnormal pulses to be low as the cardiovascular health index decreases. For example, when the user's cardiovascular health index is low even though the threshold value for the accumulated number of abnormal pulse occurrences is set to 10, 20, 30, and 50 times, the processor 310 may adjust the threshold value for the accumulated number of abnormal pulse occurrences to 10, 20, 30, and 40 times, and may most strongly respond even though the accumulated number of abnormal pulse occurrences is only 40 times, thereby preventing dangerous situations.

In an embodiment, when an irregular signal is measured, the processor 310 may determine that the electrode is not in contact with the user's body or there is a problem with a device.

When it is determined that the electrode is not in contact with the user's body, the processor 310 may request the user terminal to relocate the electrode.

In the meantime, in some embodiments, the processor 310 may derive a condition that an abnormal pulse occurs in a user, based on a time at which the abnormal pulse is detected, heartbeat information at the corresponding time, and state information of the user, may analyze biometric information and provide the user terminal with a notification requiring attention when the condition is satisfied, may determine whether an abnormal pulse is detected, by analyzing the biometric information after providing the notification, and may also update the condition when an abnormal pulse is not detected.

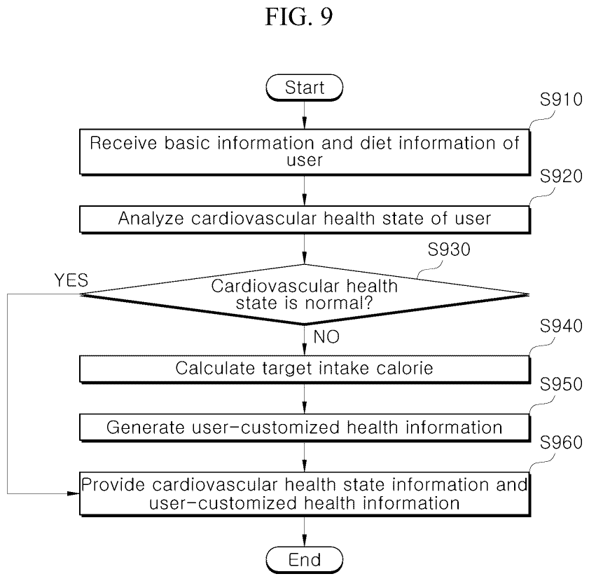

FIG. 9 is a flowchart of a method for evaluating cardiovascular conditions, according to an embodiment of the present disclosure.

A process for evaluating a cardiovascular condition according to an embodiment of the present disclosure will be described with reference to FIG. 9.

The processor 310 receives a user's basic information and diet information. (S910)

The processor 310 may receive at least one of the basic information and the diet information from the user terminal through the communication module 320.

Cardiovascular scale information includes at least one of whether an abnormal pulse occurs, blood pressure information, blood sugar information, body fat percentage information, and cholesterol level information.

The basic information includes at least one of age information, weight information, respiratory rate information, exercise information, body temperature information, information about smoking, and information about drinking.

The processor 310 analyzes the user's cardiovascular condition. (S920) The processor 310 may analyze the user's cardiovascular condition based on at least one of the cardiovascular scale information and the basic information. The processor 310 may generate user-customized health information based on the cardiovascular condition through the analysis.

In the meantime, when there is insufficient information or information, which is required additionally, during a process of analyzing the user's cardiovascular condition based on at least one of the cardiovascular scale information and the basic information, the processor 310 may request the user terminal or a guardian terminal to provide the corresponding information.

In the meantime, the processor 310 may use the trained machine learning model to analyze the user's cardiovascular condition. In some embodiments, the processor 310 may create and utilize a personalized prediction model by continuously collecting and analyzing the cardiovascular scale information and the basic information.

The processor 310 determines whether the user's cardiovascular condition is normal. (S930)

When it is determined in S930 that the user's cardiovascular condition is abnormal, the processor 310 calculates a target intake calorie. (S940)

The processor 310 generates user-customized health information. (S950)

When the analysis of the user's cardiovascular condition indicates that the cardiovascular condition is out of a normal range, the processor 310 may calculate calorie information consumed by the user based on the diet information and may calculate a target intake calorie for adjusting the user's cardiovascular scale information to the normal range based on the correlation between an intake calorie and the cardiovascular scale information.

When the user's cardiovascular condition is analyzed as being out of a normal range, the processor 310 may calculate calorie information consumed by the user based on the diet information and may calculate the target intake calorie for adjusting the user's cardiovascular scale information to the normal range based on the correlation between the intake calorie and the cardiovascular scale information.

Moreover, the processor 310 may generate customized diet information corresponding to the calculated target intake calorie as customized health information.

In this case, the processor 310 may calculate a target intake calorie for adjusting the user's cardiovascular scale information to a normal range in consideration of the user's basic information.

When a cardiovascular health state is analyzed as being out of a normal range, the processor 310 may calculate calorie information consumed by the user based on the diet information and may calculate the target intake calorie, which is the amount by which the user's intake calories should be adjusted, to adjust the user's cardiovascular scale information to the normal range based on the correlation between the intake calorie and the cardiovascular scale information.

Furthermore, the processor 310 may calculate customized exercise information corresponding to calories, which should be reduced to correspond to the calculated target intake calorie, as customized health information.

The cardiovascular scale information includes at least one of blood pressure information and blood sugar information.

In an embodiment, the processor 310 may include the user's preferred foods based on the user's age information and diet information when generating customized diet information. In an additional embodiment, the processor 310 may use the user's allergy information and the user's non-preferred food information.

In an embodiment, when generating the customized exercise information, the processor 310 may generate customized exercise information such that exercises that the user may perform is included based on the user's age information, weight information, and breathing information.

As an extension of an embodiment, the processor 310 may generate the customized exercise information by adjusting the body utilization range of the exercise content based on at least one of the user's age information, weight information, and breathing information.

The processor 310 provides cardiovascular condition information and the user's customized health information. (S960)

The processor 310 may transmit at least one of the user's cardiovascular condition information and the user's customized health information through the communication module 320 so as to be displayed on the user terminal.

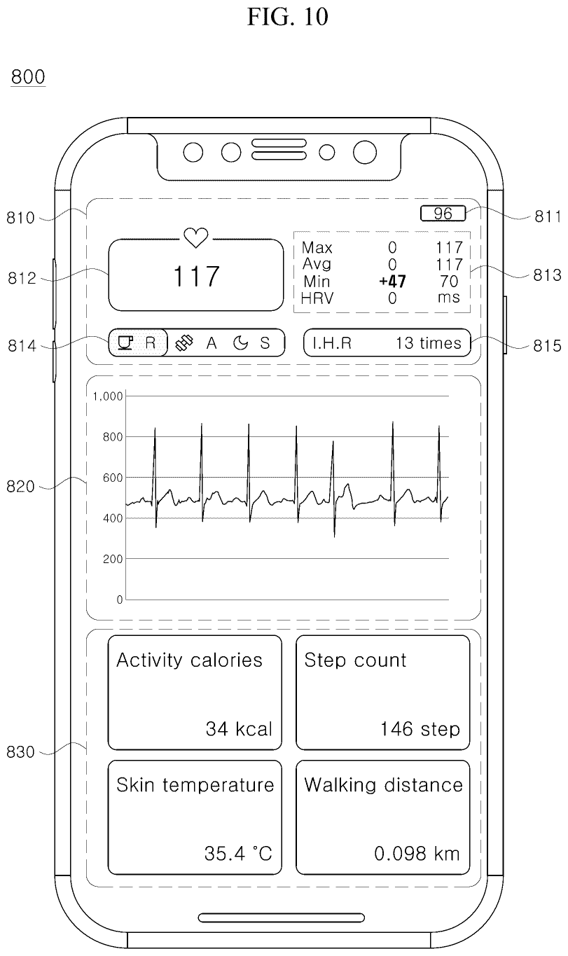

FIG. 10 is a diagram illustrating state monitoring information provided to a user terminal.

In an embodiment of the present disclosure, the electronic device 100 may communicate with the server 300, and the server 300 may provide a cardiovascular condition monitoring service and a cardiovascular condition evaluation service through a web or an app.

Referring to FIG. 10, the UI of an application is displayed on a user terminal 800, and the cardiovascular condition monitoring service is provided as an example.

Battery information 811, current heart rate information 812, pulse information 813, state information 814, and abnormal pulse count information 815 may be displayed on a first area 810 of a screen of the terminal 800.

In this case, the battery information 811 means the state-of-charge (SoC) of the battery included in the electronic device 100.

A graph of recent patterns of biometric information may be displayed on a second area 820 of the screen of the terminal 800.

At least one of the user's activity calories, step count, skin temperature, and walking distance may be displayed on a third area 830 of the screen of the terminal 800.

In an embodiment as illustrated in FIG. 10, the electronic device 100 transmits the user's biometric information measured through the sensor module 140 to the terminal 200, and the terminal 200 transmits the user's biometric information to the server 300.

Moreover, the server 300 may generate cardiovascular monitoring information based on the user's biometric information and may perform S720, S730, S740, S750, S760, and S770 of FIG. 7.

Also, the server 300 may perform this process and may transmit the generated information to the terminal 200 so as to be displayed.

Besides, the server 300 may receive cardiovascular scale information for the user, the user's basic information, and the user's diet information, may analyze the user's cardiovascular condition based on the user's cardiovascular scale information and user's basic information, and may generate user-customized health information based on the cardiovascular condition.

The server 300 may perform S910, S920, S930, S940, S950, and S960 of FIG. 9.

Also, the server 300 may perform this process and may transmit the generated information to the terminal 200 so as to be displayed.

According to an embodiment of the present disclosure, a system for monitoring cardiovascular conditions may be provided.

Furthermore, according to an embodiment of the present disclosure, a system for evaluating a user's cardiovascular condition may be provided.

In addition, according to an embodiment of the present disclosure, depending on the frequency of abnormal pulse detected from biometric information collected from the user, a rest may be recommended or a notification may be provided.

Moreover, according to an embodiment of the present disclosure, a cardiovascular health index of the user based on biometric information collected from the user may be calculated and provided.

Besides, according to an embodiment of the present disclosure, the user's cardiovascular condition may be analyzed based on the user's cardiovascular scale information and basic information, and the user's customized health information may be generated and provided based on the user's cardiovascular condition.

The method according to an embodiment of the present disclosure may be implemented by a program (or an application) and may be stored in a medium such that the program is executed in combination with a server being hardware.

The above-described program may include a code encoded by using a computer language such as C, C++, JAVA, a machine language, or the like, which a processor (CPU) of the computer may read through the device interface of the computer, such that the computer reads the program and performs the methods implemented with the program. The code may include a functional code related to a function that defines necessary functions executing the method, and the functions may include an execution procedure related control code necessary for the processor of the computer to execute the functions in its procedures. Furthermore, the code may further include a reference related code of the memory 330 on which location (address) of the internal or external memory 330 of the computer should be referenced by the media or additional information necessary for the processor of the computer to execute the functions. Further, when the processor of the computer is required to perform communication with another computer or a server in a remote site to allow the processor of the computer to execute the functions, the code may further include a communication related code on how the processor of the computer executes communication with another computer or the server or which information or medium should be transmitted/received during communication by using a communication module of the computer.

The stored medium refers not to a medium, such as a register, a cache, or the memory 330, which stores data for a short time but to a medium that stores data semi-permanently and is read by a device. Specifically, for example, the stored media include, but are not limited to, ROM, RAM, CD-ROM, magnetic tape, floppy disk, optical data storage device, and the like. That is, the program may be stored in various recording media on various servers, which the computer may access, or in various recording media on the computer of the user. Further, the media may be distributed in computer systems connected over a network such that codes readable by the computer are stored in a distributed manner.

Steps or operations of the method or algorithm described with regard to an embodiment of the present disclosure may be implemented directly in hardware, may be implemented with a software module executable by hardware, or may be implemented by a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or a computer-readable recording medium well known in the art to which the present disclosure pertains.

Although an embodiment of the present disclosure is described with reference to the accompanying drawings, it will be understood by those skilled in the art to which the present disclosure pertains that the present disclosure may be carried out in other detailed forms without changing the scope and spirit or the essential features of the present disclosure. Therefore, the embodiments described above are provided by way of example in all aspects, and should be construed not to be restrictive.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A cardiovascular health management system, the system comprising:
a communication module configured to receive biometric information of a user including information about a heartbeat; and
a processor configured to generate cardiovascular monitoring and prediction information based on the received biometric information,
wherein the processor is configured to:
analyze the biometric information and record a time at which an abnormal pulse is detected, heartbeat information at a corresponding time, and state information of the user when the abnormal pulse is detected;
provide a user terminal with a number of times that the abnormal pulse occurs during a predetermined time; and
provide a phased notification to the user terminal depending on the number of times that the abnormal pulse occurs.

2. The system of claim 1, wherein the processor is configured to:
determine a state of the user by analyzing the biometric information, wherein the state of the user is determined to be one of a resting state, an activity state, and a sleep state;
when the number of the abnormal pulse occurring during the predetermined time is greater than or equal to a predetermined count and the state of the user is the resting state or the activity state, provide a notification through the user terminal; and
when the number of the abnormal pulse occurring during the predetermined time is greater than or equal to the predetermined count and the state of the user is the sleep state, provide a notification to the user terminal and a guardian terminal of the user.

3. The system of claim 2, wherein the processor is configured to:
receive a resting heart rate of the user, which is measured while the user is at rest, and an activity heart rate of the user measured while the user is moving;
set an activity reference heart rate based on the measured resting heart rate and the measured activity heart rate; and
analyze the biometric information of the user and determine the state of the user as the activity state when a heart rate is greater than or equal to the activity reference heart rate.

4. The system of claim 1, wherein the processor is configured to:
analyze the biometric information and detect an abnormal pulse when the heartbeat is irregular; and
analyze the biometric information and provide a notification to the user terminal when a heart rate is greater than or equal to a predetermined tachycardia reference heart rate and the heart rate is less than or equal to a predetermined bradycardia reference heart rate, and
wherein when the heart rate is greater than or equal to the predetermined tachycardia reference heart rate but the user is in an activity state, and the heart rate is less than or equal to the predetermined bradycardia reference heart rate but the user is in a sleep state, not provide a notification to the user terminal.

5. The system of claim 1, wherein the processor is configured to:
derive a condition that the abnormal pulse occurs in the user, based on the time at which the abnormal pulse is detected, the heartbeat information at the corresponding time, and the state information of the user;
analyze the biometric information and provide the user terminal with a notification requiring attention when the condition is satisfied;
determine whether the abnormal pulse is detected, by analyzing the biometric information after providing the notification, and update the condition when the abnormal pulse is not detected.

6. The system of claim 1, wherein the processor is configured to:
provide the user terminal with a phased notification with respect to the abnormal pulse for a plurality of respective predetermined counts, wherein content included in a notification is changed as a number of the abnormal pulse increases;
provide the user terminal with a notification for requesting an acknowledgement that a corresponding notification is acknowledged, together with a notification for recommending a rest when the number of the abnormal pulse occurring during the predetermined time is greater than or equal to a highest count among predetermined counts; and
provide a notification to a guardian terminal of the user when not receiving an acknowledgement signal that a corresponding notification is acknowledged, from the user terminal.

7. The system of claim 6, wherein the biometric information of the user further includes information about an electrode temperature, and
wherein the processor is configured to:
provide the user terminal with the notification for requesting the acknowledgement that a corresponding notification is acknowledged, together with the notification for recommending the rest when the electrode temperature is out of a predetermined temperature range even though the number of times that the abnormal pulse occurring during the predetermined time is greater than or equal to a count less than the highest count among the predetermined counts; and
provide a notification to a guardian terminal of the user when not receiving an acknowledgement signal that a corresponding notification is acknowledged, from the user terminal.

8. The system of claim 6, wherein the biometric information of the user further includes at least one of blood pressure information, blood sugar information, body fat percentage information, cholesterol level information, age information, weight information, respiratory rate information, and exercise information as additional information,
wherein the processor is configured to:
calculate a cardiovascular health index based on the number of times that the abnormal pulse occurs during the predetermined time, and the additional information; and
adjust the highest count among the predetermined counts to be low as the cardiovascular health index decreases.

9. The system of claim 1, wherein the biometric information of the user includes cardiovascular scale information about the user, basic information of the user, and diet information of the user,
wherein the cardiovascular scale information includes at least one of whether an abnormal pulse occurs, blood pressure information, blood sugar information, body fat percentage information, and cholesterol level information, and the basic information includes at least one of age information, weight information, respiratory rate information, exercise information, body temperature information, information about smoking, and information about drinking, and
wherein the processor is configured to:
analyze a cardiovascular health state of the user based on the received cardiovascular scale information and the basic information, and generate and provide customized health information of the user based on the cardiovascular health state;
when the cardiovascular health state of the user is analyzed as being out of a normal scope, calculate information about a calorie consumed by the user based on the diet information;
calculate a target intake calorie for adjusting the cardiovascular scale information of the user to a normal range based on a correlation between the consumed calorie and the cardiovascular scale information; and
generate customized diet information corresponding to the calculated target intake calorie as the customized health information.

10. A control method performed by a processor of a cardiovascular health management system, the method comprising:
receiving biometric information of a user including information about a heartbeat;
analyzing the biometric information and recording a time at which an abnormal pulse is detected, heartbeat information at a corresponding time, and state information of the user when the abnormal pulse is detected;
providing a user terminal with a number of times that the abnormal pulse occurs during a predetermined time; and
providing a phased notification to the user terminal depending on the number of times that the abnormal pulse occurs.

11. The method of claim 10, wherein the processor is configured to:
determine a state of the user by analyzing the biometric information, wherein the state of the user is determined to be one of a resting state, an activity state, and a sleep state;
when the number of the abnormal pulse occurring during the predetermined time is greater than or equal to a predetermined count and the state of the user is the resting state or the activity state, provide a notification through the user terminal; and
when the number of the abnormal pulse occurring during the predetermined time is greater than or equal to the predetermined count and the state of the user is the sleep state, provide a notification to the user terminal and a guardian terminal of the user.

12. The method of claim 11, wherein the processor is configured to:
receive a resting heart rate of the user, which is measured while the user is at rest, and an activity heart rate of the user measured while the user is moving;
set an activity reference heart rate based on the measured resting heart rate and the measured activity heart rate; and
analyze the biometric information of the user and determine the state of the user as the activity state when a heart rate is greater than or equal to the activity reference heart rate.

13. The method of claim 10, wherein the processor is configured to:
analyze the biometric information and detect an abnormal pulse when the heartbeat is irregular; and
analyze the biometric information and provide a notification to the user terminal when a heart rate is greater than or equal to a predetermined tachycardia reference heart rate and the heart rate is less than or equal to a predetermined bradycardia reference heart rate, and
wherein when the heart rate is greater than or equal to the predetermined tachycardia reference heart rate but the user is in an activity state, and the heart rate is less than or equal to the predetermined bradycardia reference heart rate but the user is in a sleep state, not provide a notification to the user terminal.

14. The method of claim 10, wherein the processor is configured to:
derive a condition that the abnormal pulse occurs in the user, based on the time at which the abnormal pulse is detected, the heartbeat information at the corresponding time, and the state information of the user;
analyze the biometric information and provide the user terminal with a notification requiring attention when the condition is satisfied;
determine whether the abnormal pulse is detected, by analyzing the biometric information after providing the notification, and update the condition when the abnormal pulse is not detected.

15. The method of claim 10, wherein the processor is configured to:
provide the user terminal with a phased notification with respect to the abnormal pulse for a plurality of respective predetermined counts, wherein content included in a notification is changed as a number of the abnormal pulse increases;
provide the user terminal with a notification for requesting an acknowledgement that a corresponding notification is acknowledged, together with a notification for recommending a rest when the number of the abnormal pulse occurring during the predetermined time is greater than or equal to a highest count among predetermined counts; and
provide a notification to a guardian terminal of the user when not receiving an acknowledgement signal that a corresponding notification is acknowledged, from the user terminal.

16. The method of claim 15, wherein the biometric information of the user further includes information about an electrode temperature, and
wherein the processor is configured to:
provide the user terminal with the notification for requesting the acknowledgement that a corresponding notification is acknowledged, together with the notification for recommending the rest when the electrode temperature is out of a predetermined temperature range even though the number of times that the abnormal pulse occurring during the predetermined time is greater than or equal to a count less than the highest count among the predetermined counts; and
provide a notification to a guardian terminal of the user when not receiving an acknowledgement signal that a corresponding notification is acknowledged, from the user terminal.

17. The method of claim 15, wherein the biometric information of the user further includes at least one of blood pressure information, blood sugar information, body fat percentage information, cholesterol level information, age information, weight information, respiratory rate information, and exercise information as additional information,
wherein the processor is configured to:
calculate a cardiovascular health index based on the number of times that the abnormal pulse occurs during the predetermined time, and the additional information; and
adjust the highest count among the predetermined counts to be low as the cardiovascular health index decreases.

18. The method of claim 10, wherein the biometric information of the user includes cardiovascular scale information about the user, basic information of the user, and diet information of the user,
wherein the cardiovascular scale information includes at least one of whether an abnormal pulse occurs, blood pressure information, blood sugar information, body fat percentage information, and cholesterol level information, and the basic information includes at least one of age information, weight information, respiratory rate information, exercise information, body temperature information, information about smoking, and information about drinking, and
wherein the processor is configured to:
analyze a cardiovascular health state of the user based on the received cardiovascular scale information and the basic information, and generate and provide customized health information of the user based on the cardiovascular health state;
when the cardiovascular health state of the user is analyzed as being out of a normal scope, calculate information about a calorie consumed by the user based on the diet information;
calculate a target intake calorie for adjusting the cardiovascular scale information of the user to a normal range based on a correlation between the consumed calorie and the cardiovascular scale information; and
generate customized diet information corresponding to the calculated target intake calorie as the customized health information.
